(19) United States
(12) Patent Application Publication
(10) Pub. No.: US 2008/0300667 A1
(43) Pub. Date: Dec. 4, 2008

(54) SYSTEM FOR DELIVERING A STENT

(75) Inventors: Stephen Hebert, San Francisco, CA (US); Marc-Alan Levine, Pottstown, PA (US)

Correspondence Address: BERENBAUM, WEINSHIENK & EASON, P.C, 370 17TH STREET, SUITE 4800, DENVER, CO, 80202, US

(73) Assignee: Bay Street Medical, San Francisco, CA (US)

(21) Appl. No.: 12/131,792

(22) Filed: Jun. 2, 2008

Related U.S. Application Data
(60) Provisional application No. 60/932,406, filed on May 31, 2007.

(51) Int. Cl. A61F 2/84

(52) U.S. Cl. 623/1.11

(57) ABSTRACT

A stent delivery system comprising a guide having a retention member configured to exert either an internal force or an external force on the stent to assist in retaining the stent on the guide.

### CROSS-REFERENCE TO RELATED APPLICATION

This application claims priority as a Non-Provisional of U.S. Provisional Patent Application No. 60/932,406 for “System for Delivering a Stent” filed May 31, 2007, hereby incorporated by this reference in its entirety as though fully set forth herein.

### BACKGROUND

1. Technical Field

This application relates to a system for delivering a stent, and more particularly a delivery system wherein a stent is mounted on a hypotube or wire to reduce the overall profile of the system.

2. Background of Related Art

Intravascular stents are used for treatment of vascular stenosis. One type of stents are balloon expandable stents which are mounted over a balloon. Inflation of the balloon expands the stent within the vessel to dilate the stenosis. Another type of stents is self-expanding, composed of shape memory material. The self-expanding stents are compressed within a sheath and when exposed from the sheath automatically move to an expanded shape memorized position within the vessel. These stents are delivered to the area of stenosis or an aneurysm by a catheter which is inserted over a guidewire. For balloon expandable stents, the balloon is mounted on the outside of the catheter and is expanded by injection of fluid through the catheter. Expansion of the balloon expands the overlying stent. For self-expanding stents, these stents are compressed against the outer surface of the catheter and placed inside a sheath or delivery catheter or positioned inside the delivery catheter and ejected by a catheter pusher positioned inside the delivery catheter, thereby requiring a larger diameter delivery catheter.

The applicants in an earlier application recognized that utilizing a catheter with a stent mounted thereon did not enable access to small vessels. To reduce the cross-sectional dimension of the stent delivery system, the applicants developed a system for placing a stent on the guidewire or hypotube, rather than on or in the catheter (which was inserted over a guidewire), thereby eliminating the larger dimensioned catheter. This system is described in commonly assigned U.S. Pat. No. 6,989,024, the entire contents of which are incorporated herein by reference, which discloses a stent mounted on a guidewire. The stent is mounted on a reduced diameter portion of the guidewire, resulting in an overall reduced profile. Proximal and distal radiopaque marker bands, functioning as proximal and distal stops for the stent, are also described for certain embodiments.

The apparatus and method disclosed in the '024 patent is effective in accessing smaller vessels and delivering a stent to such vessels. The present application provides improvements and variations to the stent delivery systems disclosed in the '024 patent and in co-pending patent application Ser. Nos. 11/703,341 and 11/703,342, filed Feb. 7, 2007, the entire contents of which are also incorporated herein by reference. In particular, the present application provides a system that prior to fill deployment of the stent, allows retrieval or changing the position of the stent within the vessel.

### SUMMARY OF THE INVENTION

The present invention in one aspect provides a stent delivery system comprising a guide in the form of a hypotube or guidewire with a stent mounted thereon. In one embodiment, the stent is mounted on a reduced area of the guide. A flexible retention structure is positioned on the guide and applies a force to the external surface of the stent to retain it in position on the guide. Alternatively, the retention structure is positioned internal of the stent and applies a force from within the stent to press the stent against the internal wall of a delivery sheath or catheter.

The retention structure in one embodiment is made of a shape memory material having a shape memorized expanded position. The retention structure could alternatively be made of a material spring biased to an expanded (open) position.

### BRIEF DESCRIPTION OF THE DRAWINGS

Preferred embodiments of the present disclosure are described herein with reference to the drawings wherein:

FIG. 1 is a side view of a first embodiment of the stent delivery system of the present invention having a single retention arm positioned internal of the stent and shown in the expanded (open) position, the stent removed for clarity;

FIG. 2 is a side view of a second embodiment of the stent delivery system of the present invention having a two internal retention arms shown in their expanded position, the stent removed for clarity;

FIG. 3 is a side view of a third embodiment of the stent delivery system of the present invention having a single retention arm positioned internal of the stent and shown in the expanded position, the stent removed for clarity;

FIG. 4 is a side view of a fourth embodiment of the stent delivery system of the present invention having a single retention wire positioned internal of the stent, the stent shown in a partially expanded position (condition);

FIG. 5 is a side view of a fifth embodiment of the stent delivery system of the present invention having a single retention arm positioned external of the stent, the stent shown in a partially expanded position;

FIG. 6 is a side view of a sixth embodiment of the stent delivery system of the present invention having a single retention arm formed from the hypotube and positioned external of the stent, the stent shown in a partially expanded position;

FIG. 7 is a side view of a seventh embodiment of the stent delivery system of the present invention having retention arms positioned external of the stent, the stent shown in a partially expanded position;

FIG. 8A is a side view of an eighth embodiment of the stent delivery system of the present invention having retention arms with inwardly facing anchors positioned external of the stent, the stent shown in a collapsed position within the delivery sheath;

FIG. 8B is a side view similar to FIG. 8A, except showing the stent in a partially expanded position; and

FIG. 9 is a side view of a ninth embodiment of the stent delivery system of the present invention having fixed retention mechanisms at the proximal and distal end of the guidewire.

FIGS. 10A and 10B illustrate a side and sectional view of another embodiment of the present invention.

### DETAILED DESCRIPTION

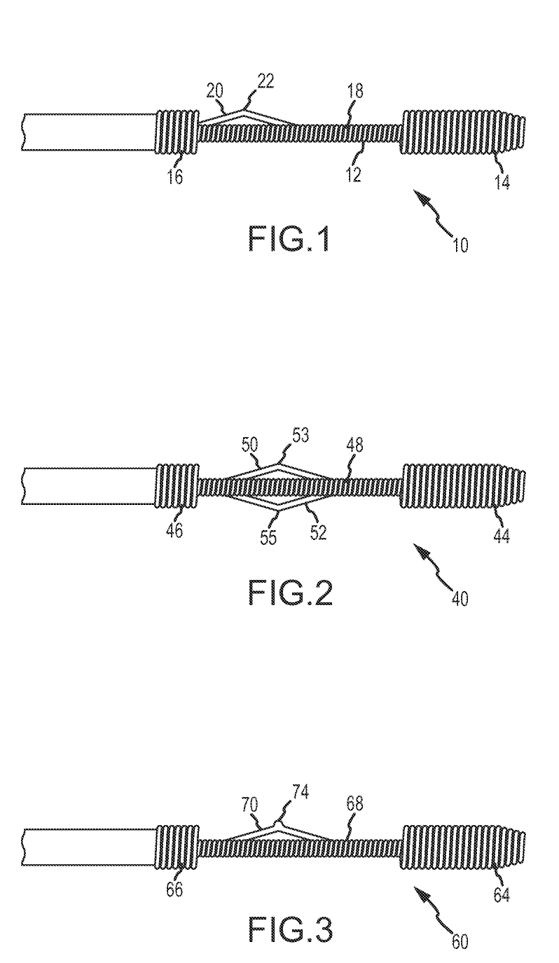

Referring now in detail to the drawings wherein like reference numerals identify similar or like components throughout the several views, several embodiments of the stent delivery system of the present invention are shown. Common to the embodiments of FIGS. 1-8 is the provision of a mechanism which allows expansion of the distal portion of the stent while maintaining a force against the proximal portion of the stent to prevent expansion of the proximal portion. In this manner, after expansion of a distal portion of the stent, the stent can be repositioned in the vessel or withdrawn back into the delivery sheath for removal from the body (or repositioning). In certain embodiments, the retention mechanism is positioned internal of the stent and applies a spring force against the stent, pressing it against the internal wall of the delivery sheath or catheter. In other embodiments, the retention mechanism is positioned external of the stent and presses the stent against the guidewire (or hypotube). In the embodiments disclosed herein, the stent is preferably positioned on a reduced profile region of the guidewire or hypotube to provide the reduced profile system to improve vessel accessibility as described in commonly assigned U.S. Pat. No. 6,989,024 and pending patent application Ser. Nos. 11/703,341 and 11/703,342, filed Feb. 7, 2007, the entire contents of each of which are incorporated herein by reference. Throughout the description reference is made to a “sheath”. In the context of the description provided herein the term “sheath” has a broad meaning and includes any member capable of being disposed over the distal segment of the guide. In some embodiments the sheath may be used to assist in retaining the stent on the guide while in other embodiments the sheath may act as a delivery device, such as a delivery catheter (e.g., microcatheter), for advancing the guide and stent to a site within the human vasculature. In some embodiments the sheath may perform both functions. In other embodiments the sheath may be used to retain the stent on the guide and is delivered with the stent through a delivery catheter to a treatment site. In such an embodiment, the sheath is removed to expose and deploy the stent after the stent has been properly positioned at the treatment site.

Turning first to the embodiments having internal engaging or retention mechanisms (FIGS. 1-4), and turning first to the embodiment of FIG. 1, stent delivery system is represented generally by reference numeral 10 and includes a guidewire 12 having a distal coil 14 and a proximal coil 16. Coils 14 and 16 may form radiopaque markers for imaging. A reduced diameter region (stepped down region) 18 of the guidewire 12 extends between the proximal and distal coils 16, 14 respectively.

A proximal end of a flexible retention arm 20 extends in a distal direction from proximal coil 16 along a length of the guidewire 12. More specifically, the proximal end of arm 20 is positioned underneath the coil 16 and can be attached thereto by conventional means such as soldering, welding or heat shrinking. The arm 20, as shown, is V-shaped in its normally biased open (expanded) configuration of FIG. 1. It is appreciated that the arm 20 may comprise any of a variety of shapes and is not limited to a V-shape. Any shape that is capable of producing a force on the internal surface of the stent may be used. The arm can be composed of spring material, including for example stainless steel, or composed of shape memory material such as Nitinol having a shape memorized position as shown. In one embodiment, the shape memory material retains its memorized positioned at an internal body temperature of a human, and thus retains the memorized position once the guide has been placed within a patient

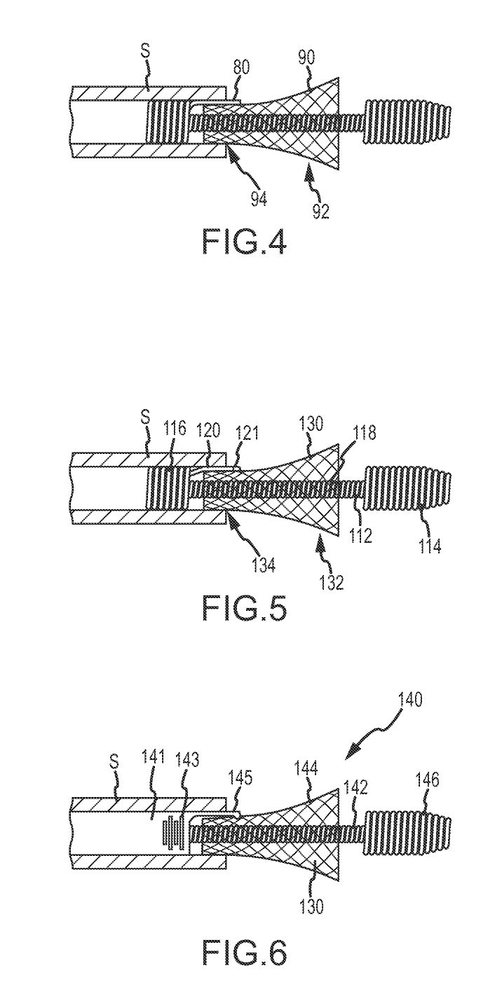

Due to the spring force of arm 20, a force is exerted on the overlying stent against the delivery sheath or catheter. This maintains the stent within the sheath. That is, a stent (not shown) is positioned on the reduced diameter region 18 of the guidewire 12 and overlies arm 20. The apex 22 of the V-arm exerts an outward force against the stent to force it against the internal wall of the delivery sheath. In this manner, when the distal portion of the stent is exposed from the sheath, the distal portion expands while the proximal portion remains trapped in the sheath by the arm 20, pinning the stent against the delivery sheath. (This aspect of partial exposure of the stent and pinning the stent against the sheath wall is shown for example in the embodiment of FIG. 4 discussed below which is applicable to the embodiment of FIGS. 1-3) At this point, the stent can be repositioned in the vessel or withdrawn back into the sheath for removal or movement to another site.

Once it is desired to place the stent, the stent is exposed from the sheath by either distal movement of the guidewire 12, proximal movement of the sheath, or relative movement of both. Once the arm 20 and stent are released from the confines of the sheath, the entire stent can expand for placement. That is, when the apex 22 of retention arm 20 is exposed from the sheath, the proximal portion of the stent is free to expand, thereby releasing the stent from the force of the retention arm 20.

In the alternate embodiment of FIG. 2, guidewire 40 has proximal and distal radiopaque marker coils 46 and 44 and a reduced diameter mounting portion 48 therebetween. Two retention arms 50, 52 are attached to the mounting portion 48 of guidewire 40 by soldering, welding or other means. In one embodiment, retention arms 50, 52 are V-shaped with respective apices 53, 55 and exert a radially outward force against the stent (not shown) in the same manner as arm 20 of the embodiment of FIG. 1, Although shown spaced from proximal coil 46, the retention arms 50, 52 (and arm 70 described below) could alternatively be positioned under or otherwise retained by proximal marker coil 46. Also, as with the other embodiments disclosed herein, alternatively, a different number of retention arms (e.g. singe or multiple) could be provided.

FIG. 3 illustrates an alternate embodiment of the stent retention system wherein a retention arm 70 extends from the reduced diameter portion (section) 68 of guidewire 60. As in FIG. 2, the reduced diameter portion 68 is formed between proximal and distal marker coils 66, 64. The V-shaped retention arm 70 differs from that of FIG. 2 in the provision of a nub or protrusion 74 at its apex. This protrusion 74 connects to the interstices of the stent (not shown) to enhance retention.

In an alternate embodiment, the retention arm could be in the form of a flexible wire 80 as shown for example in the embodiment of FIG. 4. The flexible wire 80 is spring biased to the expanded (open) position as shown to engage an internal portion of the stent, pressing it against the delivery sheath. Wire 80 can be attached in the same manner as the arms of FIGS. 1-3. FIG. 4 shows the distal portion 92 of stent 90 exposed and expanded, with the proximal portion 94 of the stent retained within the sheath S due to the force exerted by wire 80. Once wire 80 is exposed from the sheath S, the proximal portion 94 of the stent 90 can fully expand, releasing the stent 90 from the guidewire delivery system,

External mechanisms for retaining the stent are shown in FIGS. 5-8. These structures engage an external surface portion of the stent to press it against the guidewire or hypotube.

Turning first to FIG. 5, delivery system 100 includes a guidewire 112 having a radiopaque distal coil and proximal coil 114, 116, and a reduced diameter stent mounting portion 118 therebetween. A flexible arm 120 presses the stent region against the guidewire 112. That is, the arm 120, preferably formed of the material described above with respect to arm 20 of FIG. 1, is spring biased (or shape memorized) to an open or expanded position. However, within the confines of sheath S, it is forced radially inwardly to a collapsed or closed position to press against the external surface of the stent 130. Arm 120 is shown with a substantially linear portion 121 however alternatively it could be curved.

When the distal portion 132 of the stent is exposed from sheath S, the stent remains partially expanded as the proximal portion is held by arm 120. This allows for repositioning or removal. Once arm 120 is exposed from sheath S, it moves to its expanded (open) position, freeing the proximal portion 134 of stent 130 so the entire stent can expand.

FIG. 6 discloses an alternate embodiment of an external stent engaging (retention) mechanism. Stent delivery system 140 has a hypotube 141 and a guidewire 142 extending distally from the hypotube 141 and soldered or welded thereto. (Note alternatively, instead of being attached to the distal end of the hypotube, the guidewire could extend through a portion of the lumen of the hypotube, and even the entire length) The hypotube 141 may have slits 143 cut into the tube to increase flexibility. A retention arm 145 extends integrally from the hypotube 141 in a distal direction to overlie stent 130. A reduced diameter region 148 is formed on guidewire 142 proximal of distal coil 146. The distal end surface of hypotube 141 can function as a proximal stop for the stent. A proximal marker could also be provided. Flexible retention arm 145 is biased (or shape memorized) to the open position and retained in a closed or collapsed position by sheath S. Arm 145 functions in the same manner to retain the stent as flexible arm 120 of FIG. 5.

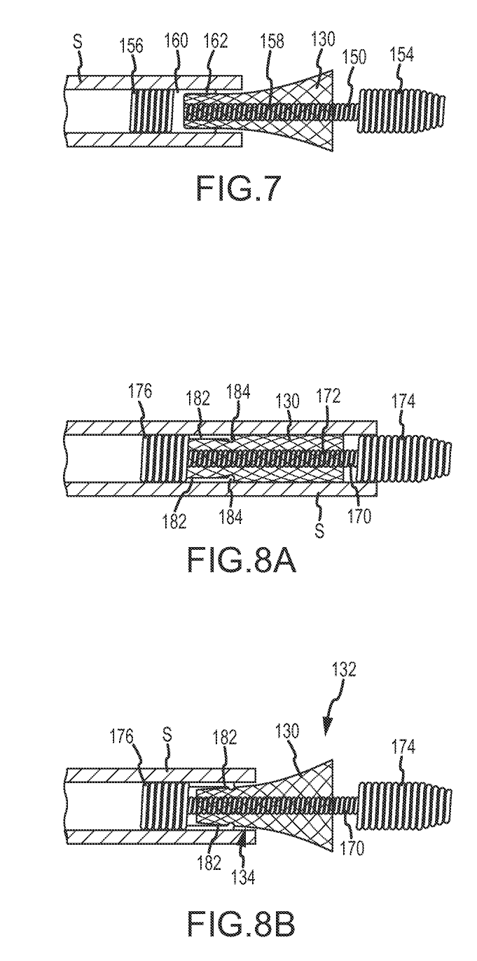

In the embodiment of FIG. 7, a ring 160 with arms 162 extending distally therefrom is attached to the proximal coil 156 of guidewire 150. Alternatively, the ring 160 could be attached to the guidewire 150. Arms 162 are preferably formed by cuts in the ring 160 forming a C-cup. Stent 130 is positioned on the reduced diameter region 158, formed between proximal marker coil 156 and distal coil 154. The arms 162 function in a similar manner to arms 121 of FIG. 5 as they engage the external surface of the stent to press it against the guidewire 150 and return to their biased open position when exposed from the sheath S. As with the other embodiments described herein, the aims 162 could alternatively be made of shape memory material such as Nitinol with a shape memory open (expanded) position. A singe arm or a plurality of arms could be provided.

FIGS. 8A and 8B disclose a stent delivery system having arms 182 with projections 184 at the tips facing radially inwardly to engage an external portion of the stent. The arms 184 can be attached to proximal marker coil 176 or retained under the coil 176. Arms 182 could also be part of a ring as in FIG. 7. Stent 130 is positioned on a reduced diameter portion 172 of the guidewire 170 between proximal marker coil 176 and distal coil marker 174.

The stent 130 is shown fully contained within the sheath in FIG. 8A. Upon retraction of the sheath S or advancement of the guidewire 170 (or movement of both in opposite directions), the distal portion 132 of stent 130 is exposed, enabling it to expand as shown in FIG. 8B. The proximal portion 134 of stent 130 is retained by the arms 182. When fully exposed, arms 182 are no longer restrained by sheath S and return to their normally open (expanded) position to enable the entire stent 130 to expand.

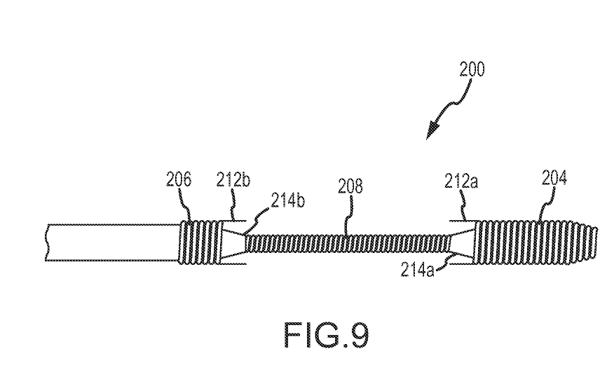

In the embodiment of FIG. 9, a different approach is utilized to retain the stent. The system 200 has two pairs of fixed arms 212a, 212b which cooperate with proximal and distal chamfered surfaces 214a, 214b to restrain the stent (not shown). That is, the distal portion of the stent is wedged between distal arms 212a and chamfered surface 214a and the proximal portion of the stent is wedged between proximal arms 212b and chamfered surface 214b. The stent is mounted on the reduced diameter portion 208 of the guidewire between proximal and distal coils 206, 204. In use, when a sufficient portion of the stent is exposed from the sheath and a sufficient middle portion of the stent expands, the stent will slide out from the distal and proximal arms 212a, 212b to expand.

Note in the embodiments disclosed herein, the guidewire can be attached at a proximal end to a distal end region of the hypotube 12 by soldering to the inside wall of the hypotube, by welding or other attachment means or alternatively a guidewire itself (without) a hypotube or a hypotube itself with a reduced diameter stent mounting region could be provided.

In an alternate embodiment, the arms of the systems discussed above can be attached to a wire through which electric current can pass to cause a phase change in the arms. That is, a power source remote from the patient would apply electrical energy to the shape memory arms to heat the arms to cause them to move to their shape memorized expanded position to release the stent. In another alternate embodiment, the arms could be electrically detachable from the stent. That is, the arms could include protrusions, hooks, or other structure for holding the stent which forms an electrolytic joint and electrical energy could be applied to dissolve the structure (joint) so that the stent is released.

The foregoing guide can be inserted into a lumen of an already placed microcatheter or alternatively can be inserted into the microcatheter or hypotube before its placement and then the assembly inserted to the surgical site. That is, the sheath which constrains the stent can be inserted with the stent mounted hypotube/guidewire as a single system. Alternatively, the sheath can be placed in the body, and the stent mounted hypotube/guidewire delivered through the already placed sheath.

In the foregoing embodiments, the marker coils can act as proximal and distal stops for the stent or alternatively other distal and/or proximal stops either integral or attached to the guide could be provided.

The arms as disclosed herein could be made from a nitinol or other shape memory tube or sheet that is cut to the desired shape and attached to the wire or coil as described above from a proximal or distal side.

Moreover, it is appreciated that the guide need not have a reduced diameter portion for mounting the stent. In alternative embodiments the stent and retention members are positioned on a guide devoid of a reduced diameter section.

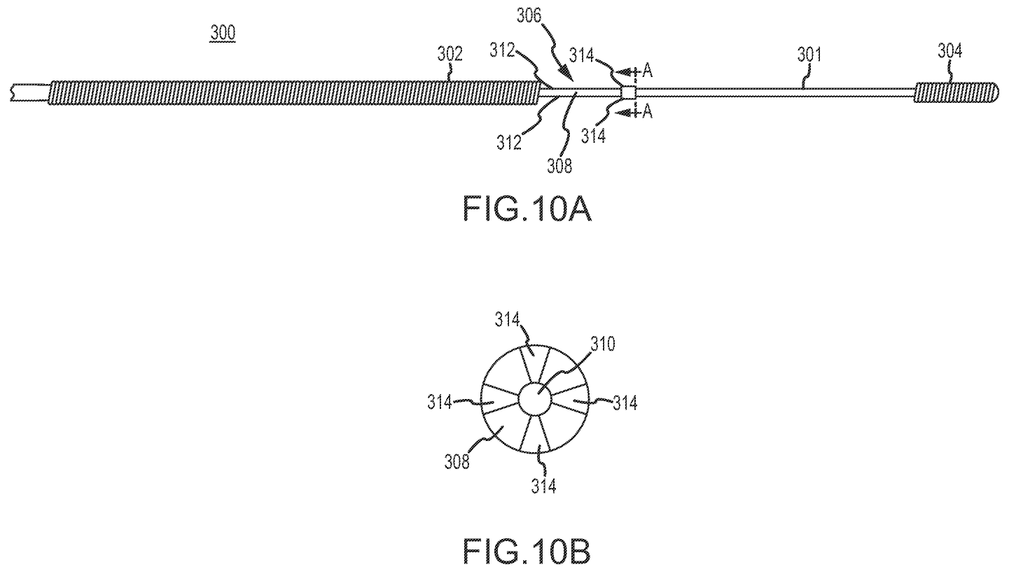

FIG. 10A illustrates a distal portion of a delivery guide 300 in an alternative embodiment. The distal portion of guide 300 includes a proximal coil segment 302 and a distal coil segment .304 positioned on the guide core 301. A device 306 for exerting a force on an internal surface of a stent (not shown) is positioned distal to proximal coil segment 302. FIG. 10B is a sectional view of device 306 along lines A-A. Device 306 includes a proximal cylindrical portion 308 having a through opening 310 that permits the device to be slid into position on the guide 300. Device 306 is secured to the guide core 301 by soldering, welding or with the use of an adhesive. Extending distally from cylindrical portion 308 are a plurality of arms 312 that have at their distal ends features 314 that are configured to contact an internal surface of a stent mounted on the guide 300. In an alternative embodiment, device 306 has only one arm 312. In practice, arms 312 are biased to urge features 314 in a direction away from guide core 301 to cause the features 314 to exert an internal force on the stent. In a preferred embodiment, device 306 is manufactured by milling and laser cutting a shape memory nickel-titanium tube and shape setting the arms 312 so that they flare outward.

While the above description contains many specifics, those specifics should not be construed as limitations on the scope of the disclosure, but merely as exemplifications of preferred embodiments thereof. For example, to provide a reduced diameter mounting region, as an alternative to a stepped region, a taper, or cut out region could be provided. Also, the tip of the wire could be shapeable. Additionally, to expose the stent, the stent mounted guidewire could be advanced from the sheath, the sheath could be retracted, or both could be moved in opposite directions. Also, other vascular prostheses can be delivered by the systems disclosed herein. Those skilled in the art will envision many other possible variations that are within the scope and spirit of the disclosure.

What is claimed is:

1. A stent delivery system comprising:
a guide having a first portion and a second portion, the first portion having a first diameter and the second portion having a second diameter less than the first diameter and forming a reduced diameter portion for receiving a stent;
a stent;
a flexible retention arm attached to the guide engaging an external proximal region of the stent to apply a force to the stent against the guide.

2. The stent delivery system as recited in claim 1, wherein the retention arm is a C-cup shape.

3. The stent delivery system as recited in claim 2, wherein the retention arm extends in a distal direction.

4. The stent delivery system of claim 1, wherein the retention arm is formed integral with the guide.

5. The stent delivery system of claim 1, wherein electrical energy is applied to arm to release the stent.

6. A stent delivery system comprising:
an elongate guide having a proximal section and a distal section;
a stent positioned on the distal section of the elongate guide;
a sheath positioned over the stent to retain the stent on the elongate guide; and
a flexible retention arm affixed to the distal end of the elongate guide and configured to engage an internal surface of the stent to apply a force to the stent against an inner surface of the sheath.

7. A stent delivery system as recited in claim 6, wherein the sheath is a delivery catheter.

8. The stent delivery system as recited in claim 6, wherein the retention arm is V-shaped.

9. The stent delivery system as recited in claim 6, further comprising a plurality or retention arms engaging internal regions of the stent.

10. The stent delivery system as recited in claim 6, wherein the guide comprises a hypotube having a proximal end, and a distal end, and a guidewire extending beyond a distal end of the hypotube.

11. The stent delivery system as recited in claim 6, further comprising proximal and distal coil regions that form a reduced diameter portion on the distal section of the elongate guide, the stent residing in the reduced diameter portion.

12. The stent delivery system as recited in claim 6, wherein electrical energy causes a phase change in the retention arm.

13. The stent delivery system as recited in claim 6, wherein the arm comprises an electrolytic joint and application of electrical energy dissolves the joint to release the stent.

14. The stent delivery system as recited in claim 6, wherein the arm comprises a shape memory material that is shape set to flare in an outward direction away from the elongate guide.

15. The stent delivery system as recited in claim 14, wherein the system comprises a plurality of flexible retention arms.

16. The stent delivery system as recited in claim 6, wherein the system comprises a plurality of flexible retention arms.
